(19) United States
(12) Patent Application Publication
(10) Pub. No.: US 2004/0015188 A1
(43) Pub. Date: Jan. 22, 2004

(54) Device for diminishing or eliminating the pain caused by a superficial therapeutic injection or superficial body tissue sampling

(76) Inventor: George Gary Coulter, Nashville, IN (US)

Correspondence Address: GEORGE GARY COULTER, 6983 GRAVEL CREEK ROAD, NASHVILLE, IN, 47448, US

(21) Appl. No.: 10/195,171

(22) Filed: Jul. 16, 2002

(51) Int. Cl. A61N 1/18

(52) U.S. Cl. 607/3; 607/46; 607/48

(57) ABSTRACT

A device is disclosed which couples the use of Trans Epithelial Nerve Stimulation with the administration of a therapeutic injection, such as an immunization, or tissue sampling procedure such as deriving a blood sample. By such an arrangement the discomfort associated with these procedures may be considerably reduced or eliminated, thereby improving compliance with a range of medical procedures. To date TENS has not been used in such an arrangement, rather development in TENS technology has been aimed at improving its efficacy. Similarly injection technology developments have been orientated around reducing cross infection risks and accidental injury to operator or patient.

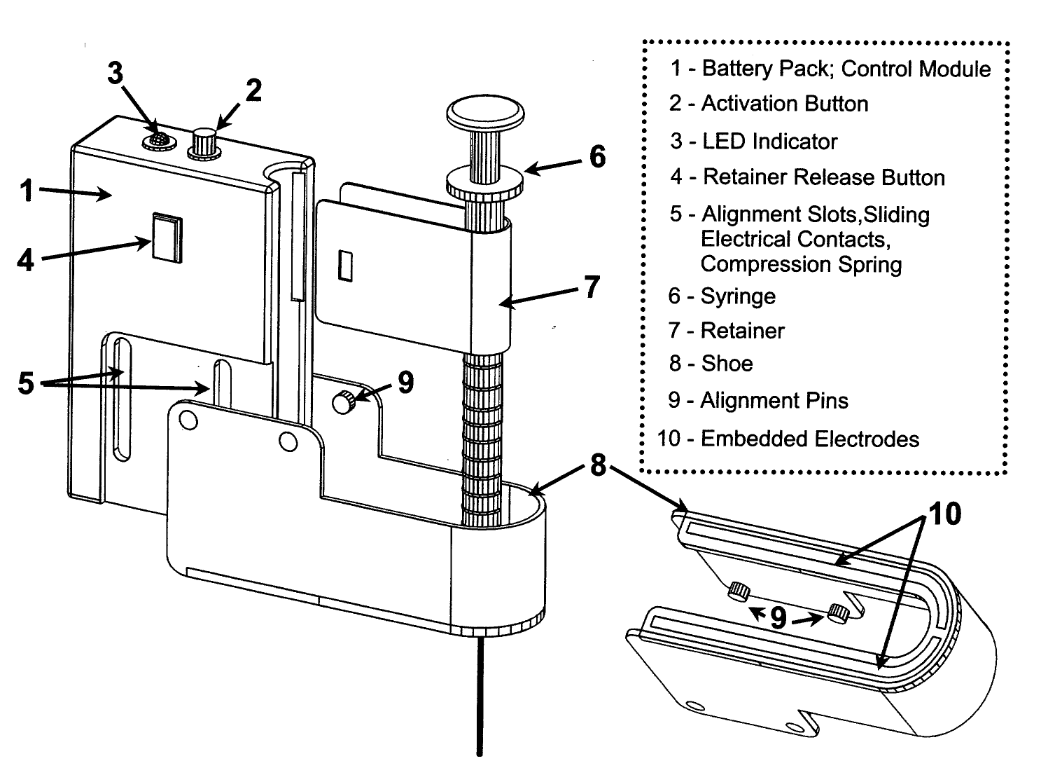

A device for diminishing or eliminating the pain caused by a superficial therapeutic injection or superficial body tissue sampling.

### CROSS REFERENCE TO RELATED APPLICATIONS

Not Applicable

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

Technology developments in the field of effecting a therapeutic injection, such as an immunization, or of a sampling of a body tissue, such as blood, have predominantly been orientated around the avoidance of cross contamination, accuracy of injection or avoidance of operator injury. Some diminution of pain of the recipient has often been a secondary goal, in part because the factors defining likelihood and extent of pain are more related to the nature of substance to be injected, site and depth of injection and the psychological expectation of pain by the patient. For the most part the common injections performed are regarded as inducing only minor and transient pain or discomfort. However, needle phobia is a significant barrier in many therapies, for example compliance with regimens for diabetes control are sometimes not adhered to because of the discomfort inherent in blood glucose testing and or insulin injection. These concerns are addressed here with a device which ameliorates or eliminates pain associated with these superficial but common medical procedures.

### SUMMARY OF INVENTION

This invention provides a means of reducing or eliminating pain on injection of therapeutic substances, by the application of TENS (trans-epithelial nerve stimulation) before during and after an injection. To date, TENS has been developed as a means of pain management for chronic conditions and additional apparatus has been developed for the management of acute pain. Where this acute situation is envisaged the designs are intended for use either in response to an injury or as an anaesthetic for a medical procedure which would last minutes to hours. Described here is a system whereby an injection device may be coupled to a TENS generating device and electrodes. In this novel arrangement, the TENS current may be applied in a manner that blocks the perception of pain and in addition by shielding the injection device and needle (where used), the visual triggers to pain expectation may be diminished.

### BRIEF DESCRIPTION OF DRAWINGS

Depicted is one arrangement of the elements needed for the device to work. In a separate case where the glove system, described below, is to be used then only the glove and TENS generating device are required with a suitable electrical connection between the two. In the present depiction, three main elements plus a syringe and needle or other are shown and the individual relationships of these parts with each other. The specific design of the individual parts may change, the present depiction is intended to represent a possible execution of the invention. Attention is drawn to the following areas of the drawings.

1 (labeled Battery Pack Control Module) A TENS generating device, comprising the necessary circuitry, battery(ies) and charging connections, controls and electrode contacting connections or surfaces in a suitable housing or case. On one surface of the housing is a receiving area for placement of syringe and needle or other, suitably notched and/or having a friction surface to prevent relative movement with the syringe and needle or other. The material selected for fabrication of the housing is appropriate for routine cleaning and disinfecting. The TENS generating device may have suitable controls to allow a range of settings and indicators to allowing the operator to know the appropriate performance of the circuitry and batteries.

7 (labeled Retainer) A clip or fastening device and release arrangement, in this case a suitably sprung U shaped clip, which holds the syringe and needle or other firmly to the housing of the TENS device (the control module). A release mechanism to allow replacement of the syringe and needle or other is arranged. In this depiction the arms of the U shaped clip are equal, but need not be so. Also in this depiction the clip is depicted of such a size and shape as not to fully cover the syringe and needle or other, but may be so designed so as to hide the body of the syringe etc. It is possible that this effect may alternatively be achieved by an arrangement of a Velcro strap or some similar arrangement to hold the syringe and needle or other firmly to the Control Module while also allowing easy removal and replacement of the syringe and needle or other. Alternative possibilities are that the U shaped clip is hinged to the Control Module and clips into place holding the syringe or other.

8,9,10 (labeled Shoe, Alignment pins, Embedded electrode) An electrode shoe, carrying at least two electrodes on its inferior surface to allow electrical contact with the patient's body part. This shoe, though mechanically attached to 1 (Battery Pack Control Module) is arranged so that it can slide in relationship to 1 (Battery Pack Control Module). Alignment slots and springs are provided between 1 and 8, the shoe, such that the whole shoe may move in a line parallel to the long axis of the syringe or other, and in a resting state is pushed downwards by the springs. Electrical contact is made with the output of the TENS circuitry through the alignment slots or contact surface between them and the inner surface of the shoe (and thence onwards to the patient electrode contact surface). In the depiction the shoe is roughly U shaped, but alternatives are many, for example the shoe may be box shaped open on its upper surface. Springs and guides are arranged as before, with the shoe overriding the lower portion of the Control Module (such that the Control Module moves inside the shoe like a piston). Electrodes are again embedded in the inferior aspect of the shoe and a hole is present to allow the needle or lance or other to emerge as the shoe rides up on the housing of the TENS device. Alternatively the shoe may have a hinged arrangement with the Control Module. In the resting state and for some degree of compression or movement of the shoe parallel with the axis of the syringe or other, the relationship of the shoe and the needle or lance is such that the needle or lance do not immediately contact the patient. This allows a controlled period of time for a TENS current to flow, producing counter irritation and nerve blockade, prior to the injection being effected. Once the shoe springs are fully compressed, with the needle or lance achieving the desired depth of entry into the patient's body part the injection can be completed by compression of the syringe plunger in the normal manner. It is envisaged that following the compression of the plunger the device may be held in situ for a short period of time allowing a continuing TENS current to flow prior to removing the entire device from the patient's body part.

### DETAILED DESCRIPTION OF THE INVENTION

A great deal of research has been conducted around the various parameters of pulse size, frequency, form, intermittancy of signal and electrode contact design that best optimize this form of pain control. Many devices are in the public domain. In the case where TENS has been proposed for acute use, it is noted that the application is clearly intended as an alternative or adjunct to the use of a pharmacological anaesthetic when a surgical intervention is envisaged and is intended to operate for some minutes (examples U.S. Pat. Nos. 6,351,674B2, 4,924,880, 5,052,391). No device utilizing TENS, however, has been considered for the avoidance of the pain expected and induced by an injection itself (where indeed the agent being injected may be an anesthetic itself or for some other therapeutic such as an immunization). Rather, development aimed at pain reduction on injection has revolved around needles injector technology or refinement of the needle systems into automatic or triggered devices either for injection or for the collection of small blood samples for example in the assessment of diabetes (examples U.S. Pat. Nos. 6,135,979, 6,102,896, 6,083,197, 5,993,412, 5,746,714). Despite these efforts, pain on therapeutic injection or sampling remains an ongoing and potentially avoidable issue. A system is described here whereby these issues can be ameliorated or removed, using a device which carries both needle and syringe or needle-less injector or blood lance and a means for delivering TENS. TENS is a well recognized method of effecting either counter irritation or nerve blockade and is widely used as a sole or adjunct method for pain relief. It does suffer from limitations in the depth and range of relief that can be afforded. Often the particular settings of the TENS current may need to be modulated over time, however for the period of relief required for an injection such as an immunization, a wide range of settings are effective. Many specific settings are in the public domain and it is envisaged here that the device may have the option for varying the parameters of TENS current, voltage, wave form, frequency etc, such that some variability per patient and operator intervention is allowed. Further, the discomfort experienced during such an injection is in part related to the psychological state of the recipient, significant needle phobia at one extreme versus the realistic expectation of pain on seeing the needle at the other. By shielding the syringe and needle (if used) from view in addition to the counter irritant nature of TENS provided by the device prior to the injection, the psychological expectation of pain can be diminished. Several layouts of electrodes are envisaged, ranging from a separate glove with electrode surfaces on the palm and digits to those directly incorporated into the TENS generator which also functions as injection carrying device. It is anticipated that if the full device is to be used, the prepared injection syringe is attached to the device, in such a way that the needle is shrouded by the electrode foot or carrier. The electrode surfaces, which may be moveable and sprung to the device, are brought into contact with the patient's skin at the site of injection and a TENS current is allowed to flow. After a short duration the device is pressed downward onto the patient (the electrode being spring loaded and allows such a movement) and in so doing the needle penetrates the skin to the desired depth for injection (intra dermal, subcutaneous or intra muscular). The plunger of the syringe is the pressed in the usual fashion or automatic actuating mechanism triggered effecting the injection, following which the entire device is removed from the patient or may be held in place for a short duration. It is envisaged that the system would also work for automatic injection devices where the injection itself is effected by some spring loaded or other mechanism. In this case the automatic injector device would be located where needle and syringe were previously located.

If the glove electrode is to be used for the application of the TENS current (either as a pain relief application in some other context or during an injection) the glove electrodes would be appropriately attached to the TENS signal generating device. The arrangement of electrode surfaces on the palmar aspect of the glove, they would be electrically isolated from each other and their connection to the TENS generating device would be arranged such that current would flow around the injection site. Thus for example the Thumb electrode on the glove could be connected to function as one contact and the other digits as the second contact, or any appropriate combination thereof. A glove electrode device was described in U.S. Pat. No. 5,070,862, where the electrode covered the entire palmar surface, this entire area of the glove acting as one of the two necessary electrodes. Further the intent of U.S. Pat. No. 5,070,862 is for electro-massage. It is envisaged that the injection would occur in the usual manner with the digits of the gloved hand allowing electrode contact around the injection site. It is anticipated if some electrical contact medium is required to increase the electrical contact between electrodes and skin then it would be so positioned as not to be in the direct line of needle puncture site. For the most part a small quantity of sterile saline would suffice as a skin to electrode contact aid. There is much literature on the specific features of the electrical signal form, size, frequency and duration, the device envisaged here may have the facility for multiple settings which may be controllable by either patient or operator. It is further envisaged that such a system could equally be applied to the obtaining of a blood sample as is commonly required by diabetics and others whereby a stylet or lance is used to puncture a digit or other body part. In this situation the lance or stylet/lance, or automatic device would take the location where the syringe and needle were previously located and used in the above description. Thus any superficial body puncture could be aided by this arrangement.

TENS devices have not been designed for the use described above.

I claim:

1. A therapeutic injection or sampling device and process, comprising a means of generating an electrical output Trans Epithelial Nerve Stimulating current and means of applying this to a patients body part, in association with a carrier mechanism for a variety of therapeutic injection or sampling systems, such as a needle and syringe or blood sampling lance or device.

2. A therapeutic injection or sampling device and process, comprising a means of generating an electrical output Trans Epithelial Nerve Stimulating (TENS) current and means of applying this TENS current to a patient utilizing a glove carrying electrodes on its surface, whereby the TENS current can be applied to the patient around the desired injection or sampling site.

3. A therapeutic injection or sampling device which comprises at least three connected components, a TENS generating apparatus, two or more patient electrode contacts and an injection, or sampling system, such as a syringe and needle.
